(19) United States
(12) Patent Application Publication
(10) Pub. No.: US 2005/0148922 A1
(43) Pub. Date: Jul. 7, 2005

(54) Thermoplastic composition and products made therefrom

(76) Inventors: William Grover Reeves, Appleton, WI (US); Li Fu Chen, West Lafayette, IN (US)

Correspondence Address: KIMBERLY-CLARK WORLDWIDE, INC., 401 NORTH LAKE STREET, NEENAH, WI, 54956, US

(21) Appl. No.: 10/750,258

(22) Filed: Dec. 31, 2003

(51) Int. Cl. A61F 13/00

(52) U.S. Cl. 602/52

(57) ABSTRACT

The present invention provides for a thermoplastic composition formed form at least one carbohydrate dissolved in at least one solvent at a sufficiently high concentration of carbohydrate, and a method of making the same. The thermoplastic composition can be made into products that are soft or firm, absorbent or non-absorbent, and hydrophobic or hydrophilic without the addition of wetting agents.

### BACKGROUND OF THE INVENTION

Lower denier fibers from synthetic thermoplastic polymers can be produced by a number of extrusion processes. For example, the melt-blowing extrusion process is of particular relevance to the present invention. In the melt-blowing process, molten polymers are extruded through a series of small diameter orifices into a high velocity air stream flowing generally parallel to the extruded fibers. This draws or stretches the fibers as they cool. The stretching serves two purposes. It causes some degree of longitudinal molecular orientation and it reduces the ultimate fiber diameter. These are termed “microfibers” since their diameter is generally less than 20 micrometers (approximately 1 denier). Another example of particular relevance to the present invention is the spunbonding extrusion process. The spunbond process pertains to small diameter fibers which are formed by extruding molten thermoplastic material as filaments from a plurality of fine capillaries of a spinneret having a circular or other configuration, with the diameter of the extruded filaments then being rapidly reduced by a conventional process such as that described in U.S. Pat. No. 4,340,563 to Appel et al., U.S. Pat. No. 3,692,618 to Dorschner et al., U.S. Pat. No. 3,802,817 to Matsuki et al., U.S. Pat. Nos. 3,338,992 and 3,341,394 to Kinney, U.S. Pat. No. 3,502,763 to Hartmann, U.S. Pat. No. 3,502,538 to Peterson, and U.S. Pat. No. 3,542,615 to Dobo et al., each of which is incorporated herein in its entirety by reference. Spunbond fibers are generally continuous and often have average deniers of about 0.3 or larger, more particularly, between about 0.6 and about 10.

Fibers in general can be utilized in the production of personal and medical care articles. Such fibers may include carbohydrate derivatives, such as cellulose. Cellulose is a straight-chain polymer of anhydroglucose with beta 1-4 linkages. Cellulose fiber in its natural form comprises such material as cotton; wood fibers, such as bleached Kraft softwood or hardwood, high-yield wood fibers, ChemiThermoMechanical Pulp fibers and recycled fibers; bagasse fibers; milkweed fluff fibers; wheat straw; kenaf; hemp; pineapple leaf fibers; and peat moss. Cellulose in the form of solvent spun fiber (e.g., regenerated cellulose compositions) comprises products such as rayon, cellulose acetate and lyocell. Examples of personal and medical care articles that comprise such fibers include bandages, tissues, absorbent composites, dressings, gauze, diapers, disposable training pants, incontinence articles, sanitary napkins, disposable garments, disposable surgical gowns or drapes, face masks, inserts for absorbent articles, shoe inserts, antiperspirant patches, breast pads, helmet liners, wound dressings, sterile wrap, covers for automobiles, and disposable ground covers.

While the aforementioned carbohydrate derivatives comprise advantages in regards to the production of personal care items, they also comprise disadvantages. For example, an advantage of rayon is that it can be produced as a continuous non-woven web, which is desirable for the production of personal care items. However, rayon also has very poor wet strength which results in sagging of a product, as well as loss of a product's shape when contacted with water. In contrast, cotton typically results in a highly wettable product; however, it generally cannot be produced as a continuous web. Therefore, there may be a need a cellulose composition that can be formed as a continuous web while simultaneously being highly wettable.

In general, natural cellulose exists as the cellulose I structure, while solvent spun cellulose typically exists as the cellulose II structure. These cellulose structures are known to one of ordinary skill in the art. In general, from a thermodynamic standpoint, cellulose II is the most stable form of cellulose. However, it is desirable to produce products with cellulose I characteristics, such as high wettablility. In general, cellulose regenerated from a cellulose solution favors the formation of cellulose II. Therefore, there may be a need for a cellulose composition that can be regenerated to exhibit cellulose I, cellulose II, or amorphous cellulose properties.

Luo et al. discloses in U.S. Pat. No. 6,306,334 that in the past, strong fibers of regenerated cellulose have been produced by the viscose and cuprammonium processes. In the viscose process, cellulose is first steeped in a mercerizing strength caustic soda solution to form alkali cellulose. This is reacted with carbon disulfide to form cellulose xanthate which is then dissolved in dilute caustic soda solution. After filtration and dearation, the xanthate solution is extruded from submerged spinnerets into a regeneration bath of sulfuric acid, sodium sulfate, zinc sulfate, and glucose to form continuous filaments. The result is viscose rayon. Cellulose is also soluble in a solution of ammoniacal copper oxide. This property forms the basis for production of cuprammonium rayon. The cellulose solution is forced through submerged spinnerets into a solution of five percent caustic soda or diluted sulfuric acid to form the fibers. After decoppering and washing the resulting fibers have great wet strength. Cuprammonium rayon is available in fibers of very low deniers which is ideal for personal care products.

However, in the article “A New Era with New Fibers”, Peter Lennox-Kerr discloses that one of the problems with viscose rayon is that the dissolving pulp from which fibers are made calls for high quality cellulose, such as from eucalyptus trees. This in turn results in high production costs. Therefore, in addition to the aforementioned need, there is a further need for a cellulose composition with lower production costs, such as with a low-cost source of cellulose fiber (e.g., recycled fiber). Additionally, in the article “Ecological Problems in the Preparation of Hydrocellulose Fibers”, S. P. Papkov further discloses that the viscose process generates carbon disulfide and hydrogen sulfide gases, both of which are toxic. Therefore, there may a need for a cellulose composition that is produced and regenerated with more environmentally friendly (i.e., less toxic) inputs and byproducts.

In U.S. Pat. No. 5,290,349, it is revealed that another solvent-spun fiber product, cellulose acetate, is produced when a cellulose derivative is solubilized in an organic solvent, and then spun into water or alcohol where it coagulates to form fiber. The fiber may be regenerated from its derivative form to true (nonderivatized) cellulose using an alkaline solution, but such a regeneration step is rarely performed. Therefore, there may be a need for a cellulose composition that is relatively easy to regenerate and that can produce either derivative or nonderivative cellulose based on production and regeneration factors.

In addition to the production of viscose, lyocell and cellulose acetate, U.S. Pat. No. 5,290,349 discloses that another example of a solvent-spun process involves the dissolution of true cellulose in a solvent which is then spun into a coagulation bath in which fiber formation occurs. Due to the high processing costs and the generally inferior properties of the fiber products formed when nonderivatized cellulose is employed in the solvent-spun process, derivatized cellulose is generally employed when producing solvent-spun fibers. However, the production of solvent-spun fiber by such methods are disadvantageous due to the aforementioned high capital costs and environmental considerations associated with their use. For this reason, there may be a need for modified or alternative methods for producing solvent-spun fiber.

In U.S. Pat. No. 5,290,349, it is revealed that an 85 percent phosphoric acid solution was tested for use as a cellulose solvent and was found to dissolve cellulose well. However, the resulting solution contained gels and fibers which made filtration very difficult. Additionally, when phosphoric acid was tested, the cellulose went into solution well, but the phosphoric acid could not be washed from the resulting fiber (D. M. MacDonald, The Spinning of Unconventional Cellulose Solutions in Turbak et al., “Cellulose Solvent Systems” ACS Sym. Seri. 58 (1977)). Solutions of 52.5 percent Ca(CNS)₂, DMF/N₂O₄, and DMSO/para-formaldehyde were also tested. These too proved unsuccessful for use. While these solutions were found to be acceptable cellulose solvents, they either formed weak fibers or were difficult to recover from the coagulation medium once fiber formation occurred (Hudson, S. M., Cuculo, L. A., J. Macromolecular Science Rev., Macromolecular Chemistry (1980) C18(1) p. 64). In addition, MacDonald supra also reported testing a 64 percent ZnCl₂ solution. However, as with the previous solvents, the results of that particular experiment were unacceptable. In particular the solubilized cellulose could not be spun and the coagulated fibers were noncohesive. Therefore, there may be a need for a cellulose composition which can dissolve well in a relatively low toxicity solvent; which can form an stable intermediary prior to regeneration; which forms strong, soft fibers upon regeneration; and which allows for substantial recovery of the solvent during the regeneration process.

ZnCl₂ is a solvent that may be view as having relatively low toxicity. However, its use in a solvent-spun process has until now proven impractical, enjoying very limited use as a solvent component for cellulose. For example, in the carbon fiber process, cellulose is solubilized in a mixture of ZnCl₂ and hydrochloric acid (HCl) to form a viscous solution which is then extruded into a methanol bath wherein the cellulose coagulates to form fibers. These fibers are usually weak, and while they can generally be handled with tweezers, they are not usually strong enough to permit spinning. Following coagulation, the ZnCl₂/HCl is removed from the cellulose by prolonged soaking in the methanol bath and the fibers are then carbonized. Therefore, there may be a need to utilize a relatively low toxicity solvent, such as ZnCl₂, to dissolve a carbohydrate derivative, such as cellulose, to produce a product that can be run through a spunbond or a melt-blown process as a thermoplastic intermediary, and can produce either cellulose I or cellulose II as desired after regeneration, while recovering the majority of the ZnCl₂ for future use.

### SUMMARY OF THE INVENTION

Within the context of this specification, each term or phrase below will include the following meaning or meanings:

“Degree of polymerization” refers to the number of repeating chemical units that make a single polymer chain.

“Dope” refers to any thick liquid or pasty substance formed from dissolving a carbohydrate in a solvent according to the present invention.

“Glass transition temperature” refers to the temperature at which the viscous liquid becomes relatively rigid and self-supporting.

“Hydrophilic” describes a material or surface which is wetted by aqueous liquids in contact therewith. The degree of wetting can, in turn, be described in terms of the contact angles and the surface tensions of the liquids and materials involved. Equipment and techniques suitable for measuring the wettability of particular fiber materials or blends of fiber materials can be provided by a Cahn SFA-222 Surface Force Analyzer System. When measured with this system in accordance with the procedure described in detail herein below, fibers having contact angles less than 90° are designated “wettable”, while fibers having contact angles greater than 90° are designated “nonwettable”.

“Meltblown” refers to fibers formed by extruding a molten thermoplastic material through a plurality of fine, usually circular, die capillaries as molten threads or filaments into converging high velocity heated gas (e.g., air) streams which attenuate the filaments of molten thermoplastic material to reduce their diameters. Thereafter, the meltblown fibers are carried by the high velocity gas stream and are deposited on a collecting surface to form a web of randomly dispersed meltblown fibers. Such a process is disclosed, for example, in U.S. Pat. No. 3,849,241 to Butin et al, which is incorporated herein by reference. Meltblown fibers are microfibers which may be continuous or discontinuous, are generally about 0.6 denier or smaller, and are generally self bonding when deposited onto a collecting surface.

“Non-woven” or “non-woven web” refers to materials or webs that are formed without the aid of a textile weaving or knitting process. The composite comprises individual or groups of fibers or threads which are interlaid, but not in an identifiable manner as in a knitted fabric. Non-woven composites have been formed from many processes such as, for example, meltblowing processes, spunbonding processes, and bonded-carded processes.

The term “personal and medical care articles” refers to articles such as bandages, tissues, absorbent composites, dressings, gauze, diapers, disposable training pants, incontinence articles, sanitary napkins, disposable garments, disposable surgical gowns or drapes, face masks, inserts for absorbent articles, shoe inserts, antiperspirant patches, breast pads, helmet liners, wound dressings, sterile wrap, covers for automobiles, and disposable ground covers.

“Regenerated” and “regeneration” refer to the process of washing the thermoplastic intermediate of the present invention to restore the carbohydrate to its original chemical form and to recover the solvent used in the dissolving process.

“Spunbond” refers to small diameter fibers which are formed by extruding molten thermoplastic material as filaments from a plurality of fine capillaries of a spinneret having a circular or other configuration, with the diameter of the extruded filaments then being rapidly reduced by a conventional process such as that described in U.S. Pat. No. 4,340,563 to Appel et al., U.S. Pat. No. 3,692,618 to Dorschner et al., U.S. Pat. No. 3,802,817 to Matsuki et al., U.S. Pat. Nos. 3,338,992 and 3,341,394 to Kinney, U.S. Pat. No. 3,502,763 to Hartmann, U.S. Pat. No. 3,502,538 to Peterson, and U.S. Pat. No. 3,542,615 to Dobo et al., each of which is incorporated herein in its entirety by reference. Spunbond fibers are generally continuous and often have average deniers of about 0.3 or larger, more particularly, between about 0.6 and about 10.

“Thermoplastic” describes a material that is a solid at room temperature, but which can be softened or melted and then processed as a viscous liquid at a higher temperature, and which substantially returns to a nonsoftened condition when cooled to room temperature.

In accordance with the present invention, a thermoplastic carbohydrate composition is disclosed which can be produced in a continuous web, which can maintain its shape once produced and be shipped for further processing into fibers, films, and nonwovens structures, and which is highly wettable upon regeneration.

The present invention generally provides for a thermoplastic composition that may be produced from a carbohydrate source, and a method of making the same. More particularly, the present invention generally comprises a process in which a carbohydrate is dissolved in a solvent by controlling the temperature and the concentration of the active agent in solvent. In one example of the present invention, zinc chloride (ZnCl₂) is utilized as the solvent. Prior to the present invention, the use of ZnCl₂ as a carbohydrate solvent has only enjoyed limited application as described above. Moreover, its use in the formation of a thermoplastic intermediate has until now been unmentioned.

Previous disclosures have shown how carbohydrates in the form of cellulose can be dissolved in 60-74 percent aqueous zinc chloride solution. As both the concentration of the cellulose and the molecular weight of the cellulose source increase, the viscosity of the cellulose solution also increases. This can create difficulties for the use of these solutions as coatings or in the preparation of solution extruded fiber or film. Because of the increasing viscosity, development to date has generally been with low concentrations (0.5 to three percent) of low molecular weight cellulose sources. However, such an approach increases the amount of solvent which must be recycled per pound of cellulose processed, which greatly increases the cost of commercialization. In the context of the present invention, it has been discovered that this inhibiting cost of recycling the solvent could be reduced by the utilization of more powerful equipment to process solutions of higher concentration and viscosity.

It has been further discovered that as the concentration of cellulose increases, the glass transition temperature of the solution also increases. It has been found that the glass transition temperature depends on both the concentration and the degree of polymerization of the cellulose. For example, cellulose with a DP of about 300 at a concentration of about ten percent becomes a novel thermoplastic with a glass transition temperature above ambient temperature. In accordance with the present invention, concentrations of cellulose up to twenty percent have been successfully processed and it is contemplated that even higher concentrations should be possible.

The novel intermediate thermoplastic composition of the present invention can be melted or extruded in standard commercial equipment and can be used in the production of fiber, film and non-woven structures. The thermoplastic intermediate can be regenerated by washing with a regeneration solution (e.g., water, lower aliphatic alcohols, or mixtures thereof) to recover the solvent, although it should be understood that regeneration is not a necessary step for processing the thermoplastic intermediate. For example, the thermoplastic intermediate of the invention may be produced in one facility, and then can be chipped, cut, or prepared as pellets and shipped to another facility for processing (e.g., meltblown or spundbond) and/or for recovery of the solvent (e.g., the thermoplastic intermediate can be regenerated after processing). Thus, the products of the invention can be durably hydrophobic or hydrophilic, soft or firm, absorbent or non-absorbent. By controlling the rate of cooling of the extruded carbohydrate composition, non-woven structures can be made in which the fibers are largely merged or largely separate. Additionally, by controlling the carbohydrate solution concentration and temperature during regeneration, varying products can be obtained. For example, a solution of cellulose can be regenerated in the form of cellulose I or cellulose II.

As stated above, the present invention involves dissolving a carbohydrate raw material in a suitable solvent. In one example, the invention involves dissolving cellulose to form a cellulose solution. The active agent of the cellulose solvent in this example can comprise zinc chloride (ZnCl₂) or N-methyl morpholine oxide. The cellulose solution can also comprise other organic substances such as urea and inorganic substances such as ammonia and calcium chloride. It should be understood that other carbohydrates and other solvents can be used either by themselves or in combination without departing from the scope of the invention. Other carbohydrates that can be used in the present invention include, but are not limited to, cellulose derivates, alginate, agar, algin, carrageenan, fucoidan, furcellaran, laminaran, gum Arabic, gum ghatti, gum karaya, gum tragacanth, guar gum, locust bean gum, okra gum, pectin, xanthan gum, scleroglucan gum, dextrans, or mixtures thereof. Where the term “cellulose dissolved in ZnCl₂” or similar language is used it should be understood that it is intended to be read broadly and include other suitable carbohydrates, solvents, and mixtures thereof. The terms “cellulose” and “regenerated cellulose” as used herein should be construed sufficiently broadly to encompass blends of cellulose with other natural and synthetic polymers, mutually soluble in a solvent, in which cellulose is the principal component by weight. In general, any carbohydrate is useful in the present invention due to the hydrogen bonds which form between the carbohydrates and the solution (e.g., zinc chloride).

As mentioned above, upon dissolving a carbohydrate in a solvent at high concentrations, the present invention results in a pre-regeneration thermoplastic intermediary composition that can be used to form novel products, including but not limited to fiber, film, and non-woven structures. For example, a carbohydrate meltblown layer can be produced which may be placed between two spundbond webs, which may be of a differing composition, for example polypropylene, prior to regeneration to make unique layered structures. Surprisingly, it was discovered that when the carbohydrate is cellulose, after regeneration it is strongly bonded to polymeric materials, such as polypropylene, avoiding the need for adhesives. In another example, a meltblown produced from the current invention can be in the form of an absorbent cellulose layer sandwiched between two spunbond sheets which could be used to absorb blood into the middle of a medical garment or drape preventing strike through. In yet another example, it is observed that urine tends to dissolve surfactants applied to non-wettable cover stock used in personal and medical care articles, thus adversely affecting the wicking properties of such articles.

By utilizing the highly wettable regenerated products of the present invention, the need for surfactant is eliminated and the wicking properties of such products remain intact.

In another example of the present invention, a 100-percent cellulose spunbond could be produced that is completely lint free, shed free, and highly wettable. This spunbond sheet could be used as a cover stock in personal and medical care products. In still another example, a cellulose towel could be produced by laminating multiple layers of spunbond carbohydrate. Such a towel could quickly absorb grease, such as in restaurants and auto shops, and yet be washed out and reused. In yet another example, a substantially lint-free web of the present invention could provide a low-cost replacement for woven gauze in medical applications. In still another example, chitin could be added to a cellulose/ZnCl₂ solution to produce personal care products exhibiting medicinal properties. In yet another example, starch could be added to a cellulose/ZnCl₂ solution to form a porous structure after regeneration and removal of the starch. In still another example, the thermoplastic composition of the current invention could be interacted with a textured wire to generate products comprising a predetermined pattern. It should be understood that the above noted examples are just a few of many examples of the present invention and should not be construed to be inclusive or limiting in any manner.

A number of variables may affect the dope viscosity of a carbohydrate/solvent composition. One such variable is the degree of polymerization (hereinafter “DP”) of the carbohydrate source. With respect to the concentration of dissolved cellulose utilized in the process of the present invention, in general it is desirable to use a higher concentration of cellulose since a higher concentration of cellulose enables higher cellulose throughput per orifice for a unit of time. On the other hand, it will be understood that the viscosity of a cellulose solution varies directly with the average DP of the cellulose (i.e., the higher the DP, the greater will be the viscosity of the cellulose in solution). Thus, for example, in the practice of the present invention the concentration of cellulose having a DP of 3000 will typically be about one percent while the concentration of cellulose having a DP of about 150 will typically be from about twenty-five percent to about thirty percent. Again, by way of non-limiting example, in the practice of the present invention the concentration of cellulose having a DP of from about 800 to about 1000 will typically be from about eighteen percent to about twenty percent while the concentration of cellulose having a DP of about 600 will typically be from about eight percent to about nine percent. One of ordinary skill in the pulping art will understand, however, that factors such as the temperature of the dissolved cellulose and the chemical properties of the solvent will also affect the useful concentration of dissolved cellulose. Depending upon the DP of the carbohydrate, the thermoplastic composition of the present invention could be made from a carbohydrate concentration ranging from about 0.5 percent to about sixty percent. In general, the lower the DP, the higher the carbohydrate concentration that is needed to form the thermoplastic composition of the present invention, and vice versa.

In one example of the invention, the carbohydrate may be a pulp. Such a pulp may be a high α-cellulose type, generally known as a chemical pulp, or it may be a lower grade pulp such as a post-consumer mixed office waste recycled fiber. U.S. Pat. No. 6,306,334 discloses that the α value of a pulp is a measure of the amount of α-cellulose (i.e., cellulose composed of glucose monomers) present in the pulp. In general, the higher the α value of a pulp, the higher is the amount of α-cellulose. The α value of a pulp can be determined by TAPPI test T2030M-88 which is well known to one of ordinary skill in the pulping art. In addition to α-cellulose, pulp also contains hemicelluloses which are branched, low molecular weight polysaccharides associated in the plant cell wall with α-cellulose and lignin. Hemicelluloses are formed from several different monosaccharides, such as mannose, galactose and arabinose. Therefore, pulps having a low a value contain a larger proportion of hemicelluloses compared to pulps having a high a value. High a pulps typically have an a value of greater than about to 90 percent, more typically greater than about 94 percent. Lower grade pulps (low a pulps) typically have an a value of less than 90 percent, usually in the range of from about 83 percent to about 89 percent. The ability to use lower α pulps is a major advantage of the present invention since it generally comprises less expensive raw material and requires less expensive processing.

Unlike the solvent-spun processes previously discussed, the process of the present invention enables one to use both high DP cellulose (DP ranging from about 1000 to 3000) and low DP cellulose (DP ranging from about 100 to 300). In essence, the cellulose starting material of the present invention may range in DP from about 100 to about 3000. Therefore, pulp obtained from a recycled fiber pulping process (having a DP of 1000 or less) which is generally unsuitable for use in existing solvent-spun fiber processes, may be used herein according to the teachings of the present invention. In addition, the ability to use low DP cellulose in the process of the present invention is further advantageous because low DP cellulose is generally inexpensive and abundant, potentially derivable from both municipal and agricultural wastes such as corn stalks and sugar cane bagasse.

Another benefit of the present invention is that recycled fiber may be utilized as a carbohydrate source. Recycled fiber is more fully and uniformly wetted when recovered, and does not need to be dried, which represents a cost savings. Pre-wetting of the carbohydrate increases the efficacy when in a solvent, such as ZnCl₂, because it increases the diffusion of the solvent into the carbohydrate structure. This in turn prevents gel blocking within the fibers (since the distal ends of dry fibers begin to gel preferentially when exposed to solvents). In addition, pre-wetting cellulose reduces the number of solid particles normally encountered when such a solvent is added directly to dry cellulose. The presence of solid particles is generally disadvantageous in solvent-spun processes because the solid particles tend to clog the narrow openings of the spinnerettes which are frequently employed in these processes.

Another factor to consider is the solvent composition with respect to the present invention. Additives to pure solvents may provide beneficial effects to the final product. For example, small amounts of calcium added to ZnCl₂ solvents can result in a substantial increase in strength of regenerated fiber, such as by organizing the carbohydrate structure.

Processing variables may also be a factor for consideration. Such variables include, but are not limited to, extrusion head temperature, air temperature, air velocity, the mass ratio of air to dope, dope throughput rate, orifice configuration and the temperature profile along the orifice, and regeneration procedure. Other variables relate to width of the extrusion head nosepiece (i.e., the distance from nozzle centers to the air exit ports), width and configuration of the air exit ports and angle of the air stream relative to the centerlines of the nozzles. The term “orifice configuration” refers not only to the orifice itself but includes any lead in capillary section.

It is important to note that solution spinning processes of the prior art (e.g., U.S. Pat. No. 6,306,334) are quite different from the thermoplastic process of the present invention, principally in that the material from a solution process is a liquid and must be regenerated immediately, otherwise the form imposed by the extrusion process will be lost. In contrast, a thermoplastic is a solid at room temperature and the material, once cooled back to ambient temperature, will keep its shape. This allows a thermoplastic material to be formed into a structure before regeneration, something which is impossible for solution processed materials.

Another advantage of the present invention is the ability to form blends of carbohydrates and other materials. For example, starch may be blended with a zinc chloride solution of cellulose to form a structure that can be leached to create a porous structure. In another example, chitin may be blended with a solution of cellulose to produce personal and medical care items. Chitin is a polysaccharide that forms part of the hard outer integument of insects, arachnids, and crustaceans. Chitin has numerous uses, including use as a flocculating agent for wastewater; a wound healing agent; a thickener and stabilizer for foods and pharmaceuticals; an ion-exchange resin; a membrane for chromatography and electro dialysis; a binder for dyes, fabrics, and adhesives; and a sizing and strengthening agent for papers. Because of its anti-microbial activity and wound healing properties, chitin is desirable when making personal and medical care products. In general, any carbohydrate, such as starch, chitin and cellulose, is useful in the present invention due to the hydrogen bonds which form between the carbohydrates and the solution (e.g., zinc chloride). Other carbohydrates that can be used in the present invention include, but are not limited to, cellulose derivates, alginate, agar, algin, carrageenan, fucoidan, furcellaran, laminaran, gum Arabic, gum ghatti, gum karaya, gum tragacanth, guar gum, locust bean gum, okra gum, pectin, xanthan gum, scleroglucan gum, dextrans, or mixtures thereof.

When cellulose is used in the present invention as the carbohydrate, it can be regenerated by washing with water, and the zinc chloride can be recovered for reuse. Once regenerated, the thermoplastic cellulose composition formed from the present invention becomes durably hydrophilic and does not require the addition of any further wetting agent. Additionally, it will not lead to any residues which would lower the surface tension of a wetting liquid thus improving wicking within absorbent products.

The present invention is advantageous in that it teaches, in one example, the use of ZnCl₂ as a cellulous solvent in a thermoplastic process, as well as teaching the production of a thermoplastic cellulosic composition that can be processed prior to regeneration. An advantage of ZnCl₂ is that it is less toxic and less corrosive than previously employed solvents and is easily recoverable for reuse. The carbohydrate raw materials can be relatively inexpensive (e.g., recycled fiber) compared to the highly purified dissolvable grade wood fiber required by the prior art. Additionally, the products made from the pre-regenerated thermoplastic composition can be cotton-like, are highly wettable and can be manufactured as a continuous process. These and other features and advantages of the present invention will become apparent after a review of the following detailed description of the disclosed embodiments.

### DETAILED DESCRIPTION

The present invention is directed towards a pre-regeneration carbohydrate thermoplastic composition and a method of making the same.

The present invention generally comprises a process in which a carbohydrate is dissolved in a solvent at a sufficiently high concentration to form a thermoplastic intermediary. The novel intermediary thermoplastic composition of the present invention can be melted or extruded in standard commercial equipment and can be used in producing fiber, film and non-woven structures. The thermoplastic composite can be regenerated by washing with a regeneration solution (e.g., water, lower aliphatic alcohols, or mixtures thereof) to recover the solvent for re-use, although it should be understood that regeneration is not a necessary step for processing the thermoplastic composite. For example, the thermoplastic composite may be produced in one facility, and then can be chipped, cut, or prepared as pellets and shipped to another facility for processing (e.g., meltblown or spundbond) and/or recovery of the solvent. Thus, products of the present invention can be durably hydrophobic or hydrophilic, soft or firm, absorbent or non-absorbent. By controlling the rate of cooling of the extruded carbohydrate composition, non-woven structures can be made in which the fibers are largely merged or largely separate. Additionally, by controlling the carbohydrate solution concentration and temperature during regeneration, varying product properties can be obtained. For instance, a solution of cellulose according to the present invention can be regenerated in the form of cellulose I or cellulose II. In one example of the present invention, a thermoplastic cellulosic composition is disclosed which can be produced as a continuous web that is highly wettable upon regeneration.

The method of the present invention provides that a carbohydrate source is dissolved in a suitable solvent. In one example, the method provides that the carbohydrate be pre-wetted with a wetting agent. The carbohydrate source is pre-wetted to minimize gel blocking when the carbohydrate is added to an aqueous solution. In one example, the wetting agent is water. In another example, the carbohydrate is pre-wetted with a 1:1 ratio of carbohydrate to the wetting agent.

In accordance with the present invention, any carbohydrate may be used. In one example, the carbohydrate source is cellulose. The cellulose can have a DP in the range of 100 to 3000. One example of cellulose used in the invention can be found under the trade name CR 2054 pulp made by Kimberly-Clark Corp. of Dallas, Tex. which has a DP of approximately 2000. In another example, the cellulose is EXCEL 110 pulp available from Functional Foods of Elizabethtown, N.J. which has a DP of approximately 1000. In another example, the cellulose is AVICEL PH101, available from FMC Corporation, Philadelphia, Pa. which has a DP of approximately 100. In another example, the cellulose is HP 11 available from Buckeye Technologies, Inc. of Memphis, Tenn. with a DP of approximately 2000. In still another example, the cellulose source is mixed office waste recycled fiber with a DP of approximately 300 to 1000.

The final amount of the carbohydrate in the mixture may vary. In one example, the mixture has a final carbohydrate concentration in the range of from about 10 to about 35 percent by weight, and a final aqueous ZnCl₂ concentration in the range of from about 60 to about 75 percent by weight. In another example, the mixing is performed at a temperature of about 200° F., more preferably from about 100 to about 190° F., most preferably from about 145 to about 175° F.

The carbohydrate solution may then be passed through an extruder to ensure that the carbohydrate is fully dissolved, at which point a thermoplastic composition is produced. In one example, the extruder is a twin screw extruder, although other extruders may be used. The solution may be passed through the extruder once or several times, depending on the desired level of dissolution. In one example, the solution is passed through the extruder several times to ensure complete dissolution.

The thermoplastic composition may be processed into film, fiber, or nonwoven structures. The thermoplastic composition may be processed on site, or may be shipped to another facility for further processing.

In one example, the thermoplastic composition was extruded into a film using a single screw extruder, although any extruder may be used. The extruder may be further equipped with a screw for processing silicon rubber. In this example, the processing temperature is preferably from about 150 to about 270° F., more preferably from about 180 to about 250° F., most preferably, from about 190 to about 230° F.

The thermoplastic films made in accordance with the present invention can be regenerated by washing with a regenerating agent. In one example, the regenerating agent is water. It is desirable to control the temperature of the regeneration in order to control the morphology of the regenerated cellulose (i.e., cellulose I, cellulose I, amorphous cellulose).

Reference will now be made in detail to examples of the present invention which are set forth below:

### EXAMPLE 1

A mixture of six (6) parts powdered HP 11 pulp and forty-four (44) parts CR 2054 pulp was pre-wetted with an equal weight of water. The mixture was fed into a two-inch READCO twin screw extruder with a screw element configuration of inlet/3FLU/blister ring/2H2FLU/discharge, available from Readco Manufacturing Company of York, Pa., and operated at a temperature of 160° F. A sixty-seven percent (67%) by weight aqueous solution of ZnCl₂ was added to the extruder at the same time as the pulp in an amount equal to five times the mass of pulp. After the extruder had filled with material and a composition of cellulose dissolved in aqueous ZnCl₂ was being extruded, carbon dioxide gas was pumped into the center of the extruder, just past the blister ring, to cause foaming of the viscous cellulose mass at the ¼ inch slit outlet. The cellulose partially foamed into thin-walled, closed-celled bubbles. The extrudate was recognized as a thermoplastic material.

### EXAMPLE 2

100% HP 11 pulp was pre-wetted with an equal weight of water. The pre-wetted pulp and a sixty-seven percent (67%) by weight aqueous ZnCl₂ solution were simultaneously added to the extruder of Example 1 at a ratio of 1 part pulp to 3 parts ZnCl₂ and processed according to the procedure described in Example 1. The extrudate was recognized as a thermoplastic material.

### EXAMPLE 3

100% HP 11 pulp was pre-wetted with an equal weight of water. The pre-wetted pulp and a sixty-seven percent (67%) by weight aqueous ZnCl₂ solution were simultaneously added to the extruder of Example 1 at a ratio of 1 part pulp to 5 parts ZnCl₂ and processed according to the procedure described in Example 1. The extrudate was recognized as a thermoplastic material.

### EXAMPLE 4

100% HP 11 pulp was pre-wetted with an equal weight of water. The pre-wetted pulp and a sixty-seven percent (67%) by weight aqueous ZnCl₂ solution were simultaneously added to the extruder of Example 1 at a ratio of 1 part pulp to 10 parts ZnCl₂ and processed according to the procedure described in Example 1. This time, the extrudate was a very viscous liquid, and not a thermoplastic material.

### EXAMPLE 5

A mixture of one (1) part powdered HP 11 pulp and one (1) part CR 2054 pulp was pre-wetted with an equal weight of water. The pre-wetted pulp mixture and a sixty-seven percent (67%) by weight aqueous ZnCl₂ solution were simultaneously added to the extruder of Example 1 at a ratio of 1 part pulp mixture to 5 parts ZnCl₂ and processed according to the procedure described in Example 1. The extrudate was recognized as a thermoplastic material.

### EXAMPLE 6

A pre-compounded mixture of eighteen percent (18%) by weight AVICEL microcrystalline cellulose and eighty-two percent (82%) of a sixty-seven percent (67%) by weight aqueous ZnCl₂ solution was extruded according to the procedure described in Example 1 at a temperature of 210° F. The extrudate was recognized as a thermoplastic material.

As shown by these examples, at sufficiently high concentrations of carbohydrate dissolved in a solvent, the present invention produces a novel thermoplastic composition. This composition may then be extruded into film, fiber, and nonwoven structures as a continuous web, and may be regenerated to form products with cotton-like properties, such as high wettability.

Accordingly, all such modifications are intended to be included within the scope of this invention, which is defined in the following claims and all equivalents thereto. Further, it is recognized that many embodiments may be conceived that do not achieve all of the advantages of some embodiments, particularly of the preferred embodiments, yet the absence of a particular advantage shall not be construed to necessarily mean that such an embodiment is outside the scope of the present invention.

When introducing elements of the present invention or the preferred embodiment(s) thereof, the articles “a”, “an”, “the” and “said” are intended to mean that there are one or more of the elements. The terms “comprising”, “including” and “having” are intended to be inclusive and mean that there may be additional elements other than the listed elements.

As various changes could be made in the above constructions without departing from the scope of the invention, it is intended that all matter contained in the above description shall be interpreted as illustrative and not in a limiting sense.

1. A composition comprising at least one carbohydrate dissolved in at least one solvent at a sufficiently high concentration of said carbohydrate wherein said composition forms a thermoplastic at room temperature.

2. The composition of claim 1 wherein said carbohydrate is cellulose, cellulose derivates, alginate, agar, algin, carrageenan, fucoidan, furcellaran, laminaran, gum Arabic, gum ghatti, gum karaya, gum tragacanth, guar gum, locust bean gum, okra gum, chitin, starch, pectin, xanthan gum, scleroglucan gum, dextrans, or combination thereof.

3. The composition of claim 2 wherein said cellulose has a DP in the range of 100 to 3000.

4. The composition of claim 2 wherein said cellulose is recycled fiber.

5. The composition of claim 1 wherein said solvent is aqueous zinc chloride.

6. The composition of claim 5 wherein the concentration of said aqueous zinc chloride is greater than fifty percent by weight.

7. The composition of claim 5 wherein the concentration of said aqueous zinc chloride is greater than sixty-seven percent by weight.

8. The composition of claim 1 wherein said solvent is N-methylmorpholine-N-oxide.

9. The composition of claim 1 wherein said carbohydrate is pre-wetted prior to addition of said solvent.

10. The composition of claim 9 wherein said pre-wetting is accomplished with equal weights of carbohydrate and water.

11. The composition of claim 1 wherein carbon dioxide gas is pumped into an extruder to cause foaming of a carbohydrate/solvent solution.

12. The composition of claim 1 wherein said composite is produced from a carbohydrate solution concentration of about 0.5 percent to about sixty percent.

13. A fiber, film, or nonwoven structure produced by extrusion of the composition of claim 1.

14. The structure of claim 13 wherein the extrusion process is spunbond or meltblown.

15. The structure of claim 13 wherein said structures are plied or laminated.

16. An article produced by the regeneration of the structure of claim 13.

17. An article produced by the regeneration of the structure of claim 13 to obtain cellulose 1, cellulose II, or amorphous cellulose properties.

18. A product in which the structure of claim 13 further comprises additional non-cellulosic nonwovens.

19. The article of claim 16 wherein said product is a personal or medical care article.

20. The article of claim 19 wherein said article comprises bandages, tissues, absorbent composites, dressings, gauze, diapers, disposable training pants, incontinence articles, sanitary napkins, disposable garments, disposable surgical gowns or drapes, face masks, inserts for absorbent articles, shoe inserts, antiperspirant patches, breast pads, helmet liners, wound dressings, sterile wrap, covers for automobiles, and disposable ground covers.

21. A method of making a composition comprising dissolving at least one carbohydrate in at least one solvent at a sufficiently high carbohydrate concentration at ambient temperature or greater to form a thermoplastic.

22. The method of claim 21 wherein said carbohydrate is cellulose, cellulose derivates, alginate, agar, algin, carrageenan, fucoidan, furcellaran, laminaran, gum Arabic, gum ghatti, gum karaya, gum tragacanth, guar gum, locust bean gum, okra gum, chitin, starch, pectin, xanthan gum, scleroglucan gum, dextrans, or combination thereof.

23. The method of claim 22 wherein said cellulose has a DP in the range of 100 to 3000.

24. The method of claim 22 wherein said cellulose is recycled fiber.

25. The method of claim 21 wherein said solvent is aqueous zinc chloride.

26. The method of claim 25 wherein the concentration of said aqueous zinc chloride is greater than fifty percent by weight.

27. The method of claim 25 wherein the concentration of said aqueous zinc chloride is greater than sixty-seven percent by weight.

28. The method of claim 21 wherein said solvent is N-methylmorpholine-N-oxide.

29. The method of claim 21 wherein said carbohydrate is pre-wetted prior to addition of said solvent.

30. The method of claim 29 wherein said pre-wetting is accomplished with equal weights of carbohydrate and water.

31. The method of claim 21 comprising the additional step of pumping carbon dioxide gas into an extruder to cause foaming of a carbohydrate/solvent solution.

32. The method of claim 21 wherein said composite is produced from a carbohydrate solution concentration of about 0.5 percent to about sixty percent.

33. A method of producing a personal or medical care article comprising the steps of:
Dissolving a carbohydrate in a solvent to form a thermoplastic composition;
Extruding said composition into a fiber, film, or nonwovens structure; and
Regenerating said structure.

34. The method of claim 33 comprising the additional step of adding previously made non-carbohydrate structures.
